(19) United States
(12) Patent Application Publication
(10) Pub. No.: US 2017/0233695 A1
(43) Pub. Date: Aug. 17, 2017

(54) Cell Culture Media Composition and Methods of Producing Thereof

(71) Applicant: Neuclone Biologics Pty Ltd, Eveleigh, New South Wales (AU)

(72) Inventor: Noelle Sunstrom, Eveleigh (AU)

(21) Appl. No.: 15/585,005

(22) Filed: May 2, 2017

Related U.S. Application Data
(62) Division of application No. 14/773,299, filed on Sep. 4, 2015.

(30) Foreign Application Priority Data
Mar. 8, 2013 (AU) 2013900809

(51) Int. Cl. C12N 5/00; C12N 5/071

(52) U.S. Cl. CPC C12N 5/0037; C12N 5/0682; C12N 2500/90; C12N 2501/305; C12N 2502/99; C12N 2502/243

(57) ABSTRACT

A serum free cell culture media, wherein the media is adapted to be conditioned by culturing a first set of eukaryotic cells in the media, wherein the first set of eukaryotic cells use an expression vector to excrete levels of desired complex proteins into the media; wherein said desired complex proteins include human Growth Hormone (hGH), Growth Hormone-like growth factors, insulin-like growth factors, insulin, modified insulins, cytokines, mitogenic proteases and mixtures thereof; and wherein the media is adapted to grow a set of eukaryotic cells.

### TECHNICAL FIELD

The present invention relates to a cell growth media and its composition; and also methods of producing cell culture growth media.

### BACKGROUND

DNA transfection methods of mammalian cells often result in massive cell death. In a transfection most cells die prior to stable DNA integration of incoming plasmid DNA into the host genome.

A large proportion of the transfected cells undergo apoptosis and although some may have successfully integrated incoming DNA, they are destined to die via a programmed cell death response by the host. Those cells surviving stable integration of DNA and who manage to avoid apoptosis are cultured in media containing the appropriate selection for 1-2 weeks to allow them to expand into a population or pool of cells that have stably integrated foreign DNA in their genome. The stable pool has low genetic heterogeneity with respect to the incoming DNA. This is due to the expansion of only a few clones that actually survived transfection, DNA integration and selection.

A major cause of cell death in cultures is the lack of ingredients within the cell media to promote strong and robust cell growth. Additionally sonic strains of eukaryotic cells are relatively weak or not robust enough to allow for commercial products to he developed from these strains. An improved cell culture media may improve robustness, cell viability and survivability of less robust strains of eukaryotic cells.

Additionally, many cell culture media include human or animal serum, which is not preferred from a contamination risk.

It is an object of the present invention to address or meliorate one or more of the abovementioned disadvantages [background art list].

Any discussion of the prior art throughout the specification should in no way be considered as an admission that such prior art is widely known or forms part of common general knowledge in the field.

### SUMMARY

### Problems to be Solved

[insert discussion of background art]

It is an object of the present invention to overcome or ameliorate at one of the disadvantages of the prior art, or to provide a useful alternative.

### Means for Solving the Problem

A first aspect of the present invention provides for a serum free cell culture media, wherein the media is adapted to be conditioned by culturing a first set of eukaryotic cells in the media wherein the first set of eukaryotic cells use an expression vector to excrete levels of desired complex proteins into the media; and wherein the media is adapted to grow a set of eukaryotic cells.

Preferably, the second set of eukaryotic cells is Chinese Hamster Ovary Cells (CHO) cells. More preferably, the CHO cells are selected from the following group or strains: CHO-K1, CHO-DG44 DHFR- and CHO-S.

Preferably, the desired complex proteins are selected from the following group: human Growth Hormone (hGH), Growth Hormone-like growth factors, insulin-like growth factors, insulin, modified insulins, cytokines, mitogenic proteases and mixtures thereof.

Preferably, the media may comprise additional supplements to promote the growth of the second set of eukaryotic cells.

Preferably, the first set of eukaryotic cells is NeuCHO cells; and may be as deposited with the Cell Bank Australia located at 214 Hawkesbury Rd, Westmead, NSW, 2145, Australia and assigned deposit no. CBA20130024, or a subculture thereof.

The preferred media is used to promote transfection of the second set of eukaryotic cells.

A second aspect of the present invention provides for a cell culture media that includes a layer to feed a set of cells to be cultured, wherein the layer comprises CHO cells including an expression vector that promotes the secretion of human growth hormone (hGH) and wherein the layer secretes hGH into the media.

Preferably, said layer is formed by CHO cells seeded in single wells of microtitre plates prior to single cell cloning of a stable transfected pool.

A third aspect of the present invention provides for a method of producing a serum free cell culture media wherein the media is conditioned by culturing a first set of eukaryotic cells in the media wherein the first set of eukaryotic cells use an expression vector to excrete levels of desired complex proteins into the media; and wherein the media is adapted to grow a set of eukaryotic cells.

The second set of eukaryotic cells may be preferably CHO cells. These CHO cells tray be selected from the following group or strains: CHO-K1, CHO-DG44 DHFR- and CHO-S.

Preferably, the desired complex proteins is selected from the following group: human Growth Hormone (hGH), Growth Hormone-like growth factors, insulin-like growth factors, insulin, modified insulins, cytokines, mitogenic proteases and mixtures thereof.

Preferably, the media may comprise additional supplements to promote the growth of the second set of eukaryotic cells. The preferred first set of eukaryotic cells is NeuCHO cells, NeuCHO cells, may be as deposited with the Cell Rank Australia located at 214 Hawkesbury Rd, Westmead, NSW, 2145, Australia and assigned deposit no. CBA20130024, or a subculture thereof.

The preferred media is used or is suitable for use to promote transfection of the second set of eukaryotic cells.

In the context of the present invention, the words “comprise”, “comprising” and the like are to be construed in their inclusive, as opposed to their exclusive, sense, that is in the sense of “including, but not limited to”.

The invention is to be interpreted with reference to the at least one of the technical problems described or affiliated with the background art. The present aims to solve or ameliorate at least one of the technical problems and this may result in one or more advantageous effects as defined by this specification and described in detail with reference to the preferred embodiments of the present invention.

### BRIEF DESCRIPTION OF THE FIGURES

Embodiments of the present invention will now be described with reference to the drawings in which:

FIG. 1 is a graph depicting viable cell density plotted against time of various cell cultures; and

FIG. 2 is a set of two graphs depicting an increasing hit rate of finding a high producer clone cell line wherein various proteins are plotted against protein concentration.

### DESCRIPTION OF THE INVENTION

The most preferred embodiment of the present invention is cell culture media produced by use of modified CHO cells to secrete growth factors in the cell media to improve the potential growth characteristics of the conditioned cell culture media.

Preferably, the first embodiment of the present invention uses NeuCHO cell which are modified CHO DG44 cells that include an expression vector to secrete human growth hormone (hGH) into the cell culture media that they are used to condition.

The NeuCho cell line, deposited under the provisions of the Budapest Treaty with the Cell Bank Australia located at 214 Hawkesbury Rd, Westmead, NSW, 2145, Australia as of 4 Feb. 2013 and assigned accession no. CBA20130024, as is particularly suitable for use in pharmaceutical manufacture as described within the present application.

The advantage of using NeuCHO cells is that hGH is excreted into the cell culture media where it can be utilised by other microorganisms which are typically difficult to grow or lack suitable cell viability for in vitro growth.

One embodiment of the present invention describes the use of conditioned media from NeuCHO cell cultures to improve the efficiency of transfection in mammalian cells. NeuCHO cells secrete human-growth hormone (hGH).

Transfection efficiency is defined here as the number of cells surviving transfection, DNA integration and selection before the individual cells are allowed to expand to form a stable pool. Transfection efficiency is improved with the addition of conditioned media from NeuCHO cell cultures. The use of NeuCHO conditioned media maximizes the number of high producing clones that can be isolated from a stable transfected cell population. The method results in a population of cells with greater genetic heterogeneity which significantly increases the likelihood of identifying high expressing clones more quickly and with more certainty than conventional methods. This embodiment simplifies cell line development by enabling rapid identification, selection, isolation and collection of high-value clones. It improves cell line productivity, shortens timelines and reduces cost.

in a further embodiment of the present invention the NeuCHO cells may be utilised as feeder cell layer for improving efficiency of single cell cloning.

Single cell cloning methods are generally inefficient but may be greatly improved by the use of conditioned cell culture media as described within the present invention or embodiment. The expansion from a single cell to a culture can be improved through the use of NeuCHO cells and/conditioned media from NeuCHO cells.

In another embodiment of the present invention relates to the use of NeuCHO as feeder cells to facilitate the growth and expansion of high clones. The use of NeuCHO feeder cells increases the survival rate and number of clones that can be isolated in a single cloning procedure.

This invention relates to methods of Transient Gene Expression for the production of recombinant bio-pharmaceuticals and other desirable proteins, polypeptides and peptides using mammalian cell cultures. In particular, the methods of the invention involve the use of specially bioengineered cell culture media which maintains very high Viable Cell Densities during and after Transient Gene Expression. Cells cultured in the mentioned media have the ability to maintain high growth in cheap, reproducible, fully-defined protein-free medium.

The number of recombinant proteins used for therapeutic applications in recent years has increased dramatically, a market expected to reach approximately $70 billion by 2010 (Walsh 2006). Recombinant antibodies currently represent over 20% of biopharmaceuticals in clinical trials as highlighted by the US Food and Drug Administration (Pavlou and Betsey 2005). However, the production of recombinant proteins is itself expensive and time consuming and the biotechnology industry is already experiencing a shortage of manufacturing capacity (Garber 2001; Dyck, Lacroix et al. 2003). Thus, factors such as scale up, total annual manufacturing capacity, post translational modifications, choice of expression system for the biosynthesis of therapeutic proteins and speed of process set up need to be evaluated in order to make both upstream and downstream production of therapeutic proteins a cost-effective process (Verma, Boleti et al. 1998; Werner, Noe et al. 1998; Fischer, Drossard et al. 1999; Bulleid, John et al. 2000; Morton and Potter 2000).

The high throughput screening required in the drug discovery process has intensified the need for a rapid technique to produce milligram amounts of recombinant protein. In order to achieve this, transient gene expression technology has attracted much interest over the traditional stable expression technology. The speed of transient gene expression represents its major economic advantage over standard stable cell line development (Durocher, Perret et al. 2002; Meissner, Pick et al. 2001; Girard, Derouazi et al. 2002; Kunaparaju, Liao et al. 2005). However many transfection procedures result in massive cell death of the transfected cell line from a very early stage (within hours), which leads to a concomitant reduction in recombinant protein production. In order to counteract this problem, many cell lines are transfected in the presence of serum containing media. The use of such media however also poses other drawbacks. Front a regulatory perspective, there are concerns regarding the use of animal derived materials and the inherent possibility of introducing adventitious agents to the culture (Sunstrom, Sugiyono et al. 2000). The use of serum is also associated with high costs, batch to batch variability, and product purification difficulties associated with the use of such media (Zang, Trautmann et al. 1995). Alternatively, CHO cells can be cultured in the presence of media containing growth factors which confer protection to the cells during the transfection procedure, thereby allowing the cells to maintain high Viable Cell Densities consequently leading to concomitant increase in the expression of recombinant proteins.

The embodiments of the present results in increasing recombinant proteins, polypeptides and peptides production by utilizing defined NeuCHO Media capable of maintaining high Viable Cell Densities during Gene Expression in mammalian cells. Host cells are transfected using NeuCHO Media which contains human-growth hormone (hGH) so that mammalian cells transfected in the presence of NeuCHO Media have very high Viable Cell Densities post transfection consequently leading to increased protein production.

The embodiment may also a method for producing high levels of desired recombinant protein, polypeptide or peptide comprising the step of: culturing a mammalian host cell in NeuCHO Media wherein said media:

(i) NeuCHO Media may be used in culturing or transfecting any of those commonly used cell lines used in the art of expressing recombinant proteins, polypeptides and peptides. For example, the host cell may be a Chinese Hamster Ovary (CHO) cell line such as CHO-K1, CHO-DG44 DHFR- and CHO-S. These include both adherent and suspension cell lines.

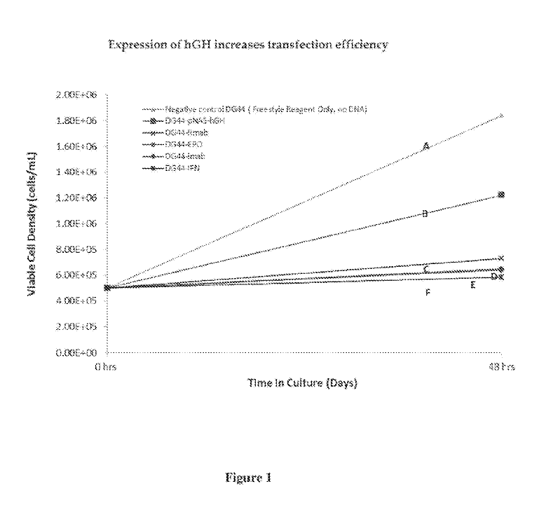

FIG. 1 is a graph depicting viable cell density plotted against time of various cell cultures. Expression of human growth hormone increases transfection efficiency by increasing survival rate of cultures following transfection. The graph represents the number of viable cells 48 hrs following transfection with plasmids encoding different recombinant proteins. DG44 cells transfected with plasmid encoding human Growth hormone gene, (DG44-hGH), has the highest viable cell density after control (no DNA) post transfection implying that hGH confers protection to DG44 cells from harsh conditions during transfection thereby leading to a higher number of cells surviving transfection. Viable Cell Density is plotted on the Y-axis in cells/mL, and the number of days in culture is plotted on the X-axis. Six line graphs are shown in the figure, namely line graph A (negative control, DG44 cells—Freestyle Reagent only, no DNA, B (DG44 pNAS-hGH or NeuCHO), C (DG44 Rmab), D (DG44-EPO), E (DG44-Imab) and F (DG44 IFN).

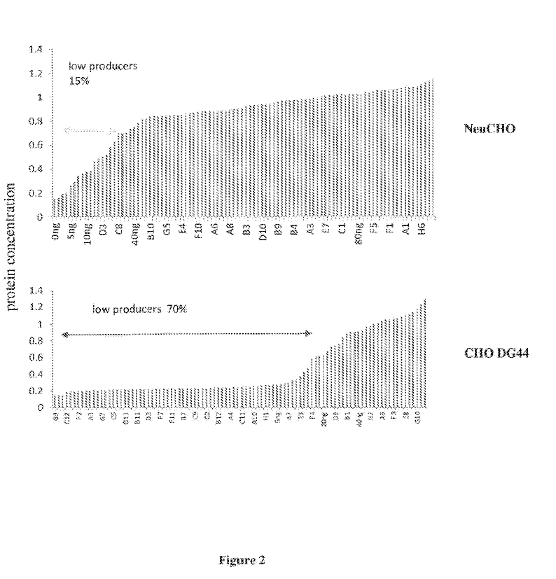

FIG. 2 is a set of two graphs depicting an increasing hit rate of finding a high producer clone cell line wherein various proteins are plotted against protein concentration. NeuCHO conditioned media or NeuCHO cells as feeder layer increases efficiency of single cell cloning. In an embodiment, NeuCHO cells were seeded in single wells of microtitre plates prior to single cell cloning of a stable transfected pool to form a layer in the cell culture media. Secretion of human growth hormone from NeuCHO cells results in an increased survival rate of single cells following Limiting Dilution Cloning.

Various additional modifications and variations are possible within the scope of the foregoing specification and accompanying drawings without departing from the scope of the invention.

Although the invention has been described with reference to specific examples, it will be appreciated by those skilled in the art that the invention may be embodied in many other forms, in keeping with the broad principles and the spirit of the invention described herein.

The present invention and the described preferred embodiments specifically include at least one feature that is industrial applicable.

### REFERENCES

Walsh, G. (2006). “Biopharmaceutical benchmarks 2006.” Nat Biotechnol 24(7): 769-76.

Pavlou, A. K. and M. J. Belsey (2005). “The therapeutic antibodies market to 2008.” Eur Pharm Biopharm 59(3): 389-96.

Garber, K. (2001). “Biotech industry faces new bottleneck.” Nat Biotechnol 19(3): 184-5.

Dyck, M. K., D. Lacroix, et al. (2003). “Making recombinant proteins in animals—different systems, different applications.” Trends Biotechnol 21(9): 394-9.

Durocher, Y., S. Perret et al. (2002). “High-level and high-throughput recombinant protein production by transient transfection of suspension growing human 293-EBNA1 cells.” Nucleic Acids Res 30(2):E9.

Girard, P., M. Derouazi et al. (2002). “100-liter transient transfection.” Cytotechnology 38(1-3):15-21.

Meissner P., H. Pick et al. (2001). “Transient gene expression: Recombinant protein production with suspension-adapted HEK293-EBNA cells.” Biotechnol Bioeng 75(2):197-203.

Kunaparaju, R., M. Liao et al. (2005). “Epi-CHO, an Episomal Expression System for Recombinant Protein Production in CHO Cells.” Biotechnol Bioeng 91(6):670-677.

Verma, R., E. Bole (1998). “Antibody engineering: comparison of bacterial, yeast, insect and mammalian expression systems.” J Immunol Methods 216(1-2): 165-81.

Werner, R. G., W. Noe, et al. (1998). “Appropriate mammalian expression systems for biopharmaceuticals.” Arzneimittelforschung 48(8): 870-80.

Fischer, R., J, Drossard, et al. (1999). “Towards molecular farming in the moving from diagnostic protein and antibody production in microbes to plants.” Biotechnol Appl Biochem 30 (Pt 2): 101-8.

Bulleid, N. J., D. C. John, et al. (2000). “Recombinant expression systems for the production of collagen.” Biochem Soc Trans 28(4): 350-3.

Morton, C. L. and P. M. Potter (2000). “Comparison of Escherichia coli, Saccharomyces cerevisiae, Pichia pastoris, Spodoptera frugiperda, and COS7 cells for recombinant gene expression. Application to a rabbit liver carboxylesterase.” Mol Biotechnol 16(3): 193-202.

Barnes, D. and G. Sato. (1980) “Methods for growth of cultured cells in serum-free medium.” Anal Biochem 102: 255-270.

Mendiaz, E., M. Mamounas, et al. (1986). “A defined medium for and the effect of insulin on the growth, amino acid transport, and morphology of Chinese hamster ovary cells, CHO-K1 (CCL61) and the isolation of insulin “independent” mutants.” In Vitro Cell Dev Biol 22: 66-74.

1.-20. (canceled)

21. A co-culture of cells comprising, a Chinese Hamster Ovary (CHO) feeder cell line comprising an expression vector encoding human Growth Hormone (hGH) and a second set of mammalian cells, wherein the CHO feeder cell line secretes hGH into the co-culture and promotes transfection of the second set of mammalian cells during co-culture.

22. A co-culture of cells according to claim 21, wherein the second set of mammalian cells are CHO cells.

23. A co-culture of cells according to claim 21, wherein the second set of mammalian cells is selected from the group consisting of CHO-K1, CHO-DG DHFR- and CHO-S.

24. A co-culture of cells according to claim 21, wherein the cells in co-culture are seeded in single wells of a microtitre plate comprising cell culture media.

25. A co-culture of cells comprising, a Chinese Hamster Ovary (CHO) feeder cell line comprising an expression vector encoding human growth hormone (hGH) and a second set of mammalian cells that produce a desired recombinant protein, wherein the CHO feeder cell line secretes hGH into the co-culture.

26. A co-culture of cells according to claim 25, wherein the second set of mammalian cells are CHO cells.

27. A co-culture of cells according to claim 25, wherein the second set of mammalian cells is selected from the group consisting of CHO-K1, CHO-DG DHFR- and CHO-S.

28. A co-culture of cells according to claim 25, wherein the cells in co-culture are seeded in single wells of a microtitre plate comprising cell culture media.

29. A method to promote transfection of a second set of mammalian cells, the method comprising co-culturing in a cell culture medium, a Chinese Hamster Ovary (CHO) feeder cell line comprising an expression vector encoding human Growth Hormone (hGH) and a second set of mammalian cells, wherein the CHO feeder cell line secretes hGH into the cell culture medium and promotes transfection of the second set of mammalian cells during co-culture.

30. The method according to claim 29, wherein the cell culture medium is serum free.

31. The method according to claim 29, wherein the cell culture medium comprises additional supplements to promote the growth of the second set of mammalian cells.

32. The method according to claim 29, wherein the second set of mammalian cells are CHO cells.

33. The method according to claim 29, wherein the second set of mammalian cells is selected from the group consisting of: CHO-K1, CHO-DG44 and CHO-S cells.
